(19) United States
(12) Patent Application Publication
(10) Pub. No.: US 2025/0041218 A1
(43) Pub. Date: Feb. 6, 2025

(54) METHOD FOR TREATING CENTRAL NERVOUS SYSTEM DISEASES

(71) Applicant: Suntec Medical, Inc., Walnut, CA (US)

(72) Inventors: Chun-Ting Cheng, Taipei (TW); Yuan-Chung Tsai, Taipei (TW); Pauline Ying Lau, Walnut, CA (US); Kuo-Liang Hou, Taipei (TW)

(21) Appl. No.: 18/919,148

(22) Filed: Oct. 17, 2024

Related U.S. Application Data
(63) Continuation of application No. PCT/US2023/018978, filed on Apr. 18, 2023.
(60) Provisional application No. 63/332,788, filed on Apr. 20, 2022.

(51) Int. Cl. A61K 9/107; A61K 31/704; A61K 45/06; A61K 47/69; A61P 25/28; A61P 35/00; C07K 16/32

(52) U.S. Cl. CPC A61K 9/107; A61K 31/704; A61K 45/06; A61K 47/6907; A61P 25/28; A61P 35/00; C07K 16/32

(57) ABSTRACT

The present invention provides a method of preventing or treating CNS diseases. The method comprises the step of administering to a subject in need thereof an effective amount of (i) a polymer-flavonoid conjugate, (ii) a flavonoid oligomer, or (iii) micelles having an outer shell comprising one or more polymer-flavonoid conjugates and optionally an inner shell comprising one or more flavonoid oligomer and a drug encapsulated within the shells. The present method brings therapeutic effective materials through blood-brain barrier to treat CNS diseases. The present method is effective to treat CNS diseases such as brain tumors, stroke, neurodegenerative diseases.

### CROSS-REFERENCE TO RELATED APPLICATION(S)

This application is a continuation of PCT/US2023/018978, filed Apr. 18, 2023; which claims the benefit of U.S. Provisional Application No. 63/332,788, filed Apr. 20, 2022. The contents of the above-identified applications are incorporated herein by reference in their entireties.

### FIELD OF THE INVENTION

The present invention provides a method of treating Central nervous system (CNS) diseases. The method comprises the step of administering to a subject in need thereof an effective amount of (i) a polymer-flavonoid conjugate, (ii) a flavonoid oligomer, or (iii) micelles having a shell formed by one or more polymer-flavonoid conjugates or one or more flavonoid oligomers, or the combination thereof, and having an agent encapsulated within the shell.

### BACKGROUND OF THE INVENTION

Central nervous system (CNS) diseases are neurological disorders that affect the structure or function of the brain or spinal cord, which collectively form the CNS. The condition may be an inherited metabolic disorder, the result of neural damages from infections, neurodegenerative conditions, stroke, brain tumor or other problems from unknown or multiple factors. CNS diseases include brain tumors, Alzheimer's disease, Parkinson's disease, Huntington's disease, migraine, infection, multiple sclerosis, addiction, arachnoid cysts, attention deficit/hyperactivity disorder (ADHD), autism, catalepsy, encephalitis, epilepsy/seizures, infection, locked-in syndrome, meningitis, migraine, myelopathy, Tourette's syndrome, Bell's palsy, headache (largest brain disease), autoimmune disorders, cerebral palsy, motor neuron disease (MND), neurofibromatosis. epilepsy and Seizures, acute spinal cord injury, amyotrophic lateral sclerosis (ALS), ataxia, Bell's Palsy, cerebral aneurysm, obsessive-compulsive disorder (OCD), defects in the cerebral cortex include microgyria, polymicrogyria, bilateral frontoparietal polymicrogyria, and pachygyria.

A brain tumor occurs when abnormal cells form within the brain. Brain tumor can be primary cancer of the brain, metastatic cancer to the brain or benign brain tumors. In general, they appear when there is a problem with cellular division. Problems with the body's immune system can lead to brain tumors.

Stroke is a medical condition in which poor blood flow to the brain causes cell death. There are two main types of stroke: ischemic, due to lack of blood flow, and hemorrhagic, due to bleeding. The main risk factor for stroke is high blood pressure. Other risk factors include high blood cholesterol, tobacco smoking, obesity, diabetes mellitus, a previous transient ischemic attack, end-stage kidney disease, and atrial fibrillation. There is no therapy to regenerate dead brain cells after stroke. The only FDA-approved drug for ischemic stroke is the tPA that breaks down blood clots in the brain. Prevention is an important clinical strategy. Oral anticoagulants such as warfarin are the mainstay of stroke prevention.

Neurodegenerative diseases are a group of diseases which primarily affect the neurons in the human brain. Examples of neurodegenerative diseases are Alzheimer's disease (AD) and other dementias, Parkinson's disease (PD) and Parkinsonism, Prion disease, Motor neuron diseases (MND), Huntington's disease (HD), Spinocerebellar ataxia (SCA) and Spinal muscular atrophy (SMA). Two major neurodegenerative diseases are Alzheimer's and Parkinson's diseases. Some neurodegenerative disorders are caused by inherited genetic changes. Majority of neurodegenerative disorders are due to a combination of genetic and environmental factors. This makes it difficult to predict who will develop disease.

Alzheimer's disease (AD) is the most prevalent neurodegenerative disease that usually starts slowly and progressively worsens. As the disease advances, symptoms can include problems with language, disorientation (including easily getting lost), mood swings, loss of motivation, self-neglect, and behavioral issues. Alzheimer's disease is believed to occur when abnormal amounts of amyloid beta (Aβ), accumulating extracellularly as amyloid plaques and tau proteins, or intracellularly as neurofibrillary tangles, form in the brain, affecting neuronal functioning and connectivity, resulting in a progressive loss of brain function. Currently, no treatments stop or reverse its progression, though some may temporarily improve symptoms.

Parkinson's disease (PD) is a long-term degenerative disorder of the central nervous system that mainly affects the motor system. It is sometimes referred to as a type of neurodegenerative disease called synucleinopathy due to an abnormal accumulation of the protein alpha-synuclein in the brain. The most obvious early symptoms of PD are tremor, rigidity, slowness of movement, and difficulty with walking. Cognitive and behavioral problems may also occur with depression, anxiety, and apathy occurring in many people with PD. Parkinson's disease dementia becomes common in the advanced stages of the disease. No cure for PD is known; treatment aims to reduce the effects of the symptoms.

The blood-brain-barrier (BBB) is a highly selective semipermeable border of endothelial cells of the central nervous system (CNS) that prevents solutes in the circulating blood vessel from non-selectively crossing into central nervous system where neurons reside. Therefore, BBB is a barrier hindering effective drugs from accumulating at brain.

There are 4 pathways for BBB penetration (passive diffusion, carrier-mediated transport, receptor-mediated transcytosis and adsorptive-mediated transcytosis). Adsorptive-mediated transcytosis (AMT) is the major pathway. AMT pathway uses Caveolae as transporting vehicle. Caveolae are a subgroup of lipid rafts present in endothelial cells of BBB. Caveolae-mediated endocytosis is a critical transporting mechanism for macromolecule uptake from blood stream to CNS.

Flavonoids have the general structure of a 15-carbon skeleton, which consists of two phenyl rings (A and B) and a heterocyclic ring (C, the ring containing the embedded oxygen).

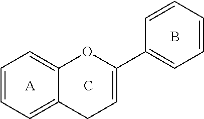

This carbon structure can be abbreviated C6-C3-C6. According to the IUPAC nomenclature, flavonoids can be classified into:
flavonoids or bioflavonoids
isoflavonoids, derived from 3-phenylchromen-4-one (3-phenyl-1,4-benzopyrone) structure
neoflavonoids, derived from 4-phenylcoumarine (4-phenyl-1,2-benzopyrone) structure

### BRIEF DESCRIPTION OF THE DRAWINGS

FIG. 1 illustrates one embodiment of a MINC (Multi-pathway Immune-modulating Nanocomplex Combination therapy)-agent, which is a micelle having a polymer-flavonoid conjugate, for example, a PEG-EGCG conjugate, in a shell and having an agent encapsulated.

FIG. 2 illustrates another embodiment of a MINC-agent, which is a micelle comprises a polymer-flavonoid conjugate, e.g., a PEG-EGCG conjugate in an outer shell and a flavonoid oligomer, for example, oligomeric EGCG (OEGCG), in an inner shell, and having an agent encapsulated.

FIG. 3 shows that more MINC-doxorubicin (PEG-EGCG, OEGCG, and doxorubicin) penetrated the surrogate BBB transwell model than doxorubicin alone.

FIG. 4 shows the tumor suppression efficacy of OEGCG in both HER2 positive and negative glioma and OEGCG overcomes temozolomide resistance.

FIG. 5 shows that comparing to the saline treatment control group, mice treated with MINC-anti-HER2 (PEG-EGCG, OEGCG, and anti-HER2) have reduced luciferase signal in A172 glioma.

FIG. 6 shows that OEGCG and MINC-BSA suppress triple negative breast cancer growth.

FIG. 7 shows the ability of OEGCG in protecting Aβ-induced cell death.

FIG. 8 shows that different polymer-flavonoid conjugate and different flavonoid oligomers all successfully generated MINC-anti-HER2 with similar particle size around 100 nm.

FIG. 9 demonstrates that different polymers in polymer-flavonoid conjugates can be used to successfully generate MINC-BSA. These different polymers are PEG (A), HA (B) and dextran (C).

FIG. 10 shows a successful formulation of MINC-anti-Aβ.

FIG. 11 shows a successful formulation of MINC-anti-α-syn.

### DETAILED DESCRIPTION OF THE INVENTION

### Definitions

The term “about” is defined as +10%, preferably +5%, of the recited value.

The term “cytokines” refer to small proteins (˜5-70 kDa) important in cell signaling. Cytokines have been shown to be involved in autocrine, paracrine, and endocrine signaling as immunomodulating agents. Cytokines include interferons, interleukins, lymphokines, tumor necrosis factors, and chemokines.

The term “epigallocatechin gallate” refers to an ester of epigallocatechin and gallic acid, and is used interchangeably with “epigallocatechin-3-gallate” or EGCG.

The term “oligomeric EGCG” (OEGCG) refers to 3-20 monomers of EGCG that are covalently linked. OEGCG preferably contains 4 to 12 monomers of EGCG.

The term “polyethylene glycol-epigallocatechin gallate conjugate” or “PEG-EGCG refers to polyethylene glycol (PEG) conjugated to one or two molecules of EGCG. The term “PEG-EGCG” refer to both PEG-mEGCG conjugate (monomeric EGCG) and PEG-dEGCG (dimeric EGCG) conjugate.

The present invention provides a method of treating CNS diseases. The method comprises the step of administering to a subject in need thereof an effective amount of (i) a polymer-flavonoid conjugate, (ii) a flavonoid oligomer, or (iii) micelles having an outer shell formed by one or more polymer-flavonoid conjugates and optionally an inner shell formed by one or more flavonoid oligomer and a drug encapsulated within the shells.

### Flavonoids

Flavonoids suitable for the present invention have the general structure of Formula I:

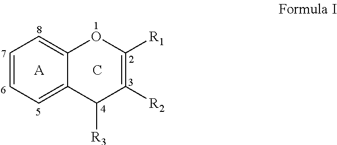

wherein:
R₁ is H, or phenyl;
R₂ is H, OH, Gallate, or phenyl; wherein the phenyl is optionally substituted by one or more (e.g., 2-3) hydroxyl;
R₃ is H, OH, or ═O (oxo); or
R₁ and R₂ together form a close-looped ring structure; or
R₂ and R₃ together form close-looped ring structure.

The 2, 3, 4, 5, 6, 7, or 8 position of Formula I, can be linked to a group containing hydrocarbon, halogen, oxygen, nitrogen, sulfur, phosphorus, boron or metals.

Examples of flavonoids of Formula I include:

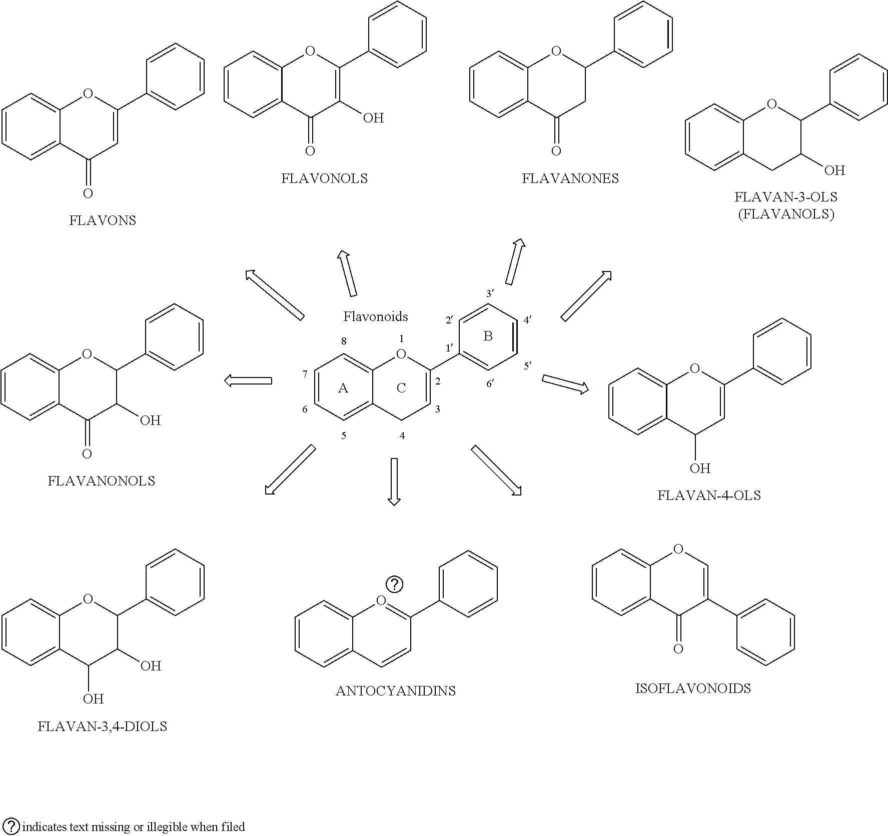

Preferred flavonoid compounds of Formula I include:

EGCG (CAS #989-51-5), EC (CAS #490-46-0), EGC (CAS #970-74-1) or ECG (CAS #1257-8-5)

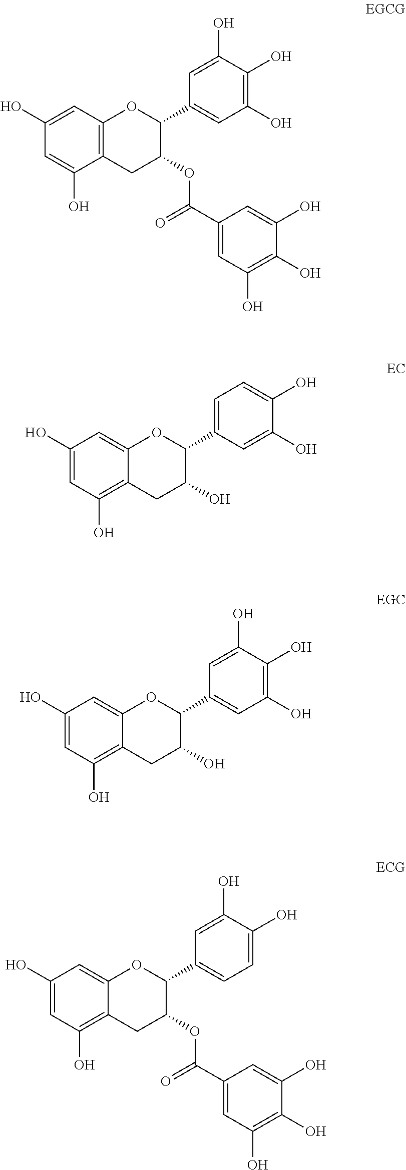

### Polymer-Flavonoid Conjugate

A polymer-flavonoid conjugate, as used herein throughout the application, refers to a conjugate of a hydrophilic polymer and the flavonoid compound of Formula I.

A hydrophilic polymer refers to a polymer that is soluble in polar solvents and can form hydrogen bonds. Hydrophilic polymers suitable for the present polymer-flavonoid conjugates include, but not limited to: poly(ethylene glycol), aldehyde-derivatized hyaluronic acid, hyaluronic acid, dextran, diethylacetal conjugate (e.g. diethylacetal PEG), D-alpha-tocopheryl polyethylene glycol succinate, aldehyde-derivatized hyaluronic acid-tyramine, hyaluronic acid-aminoacetylaldehyde diethylacetal conjugate-tyramine, cyclotriphosphazene core phenoxymethyl(methylhydrazono)dendrimer or thiophosphoryl core phenoxymethyl(methylhydrazono)dendrimer. acrylamides, oxazolines, imines, acrylic acids, methacrylates, diols, oxiranes, alcohols, amines, anhydrides, esters, lactones, terephthalate, amides and ethers polyacrylamide, poloxamers, poly(N-isopropylacrylamide), poly(oxazoline), polyethylenimine, poly(acrylic acid), polymethacrylate, poly(ethylene glycol), poly(ethylene oxide), poly(vinylalcohol), poly(vinylpyrrolidinone), polyethers, poly(allylamine), polyanhydrides, poly(β-amino ester), poly(butylene succinate), polycaprolactone, polycarbonate, polydioxanone, poly(glycerol), polyglycolic acid, poly(3-hydroxypropionic acid), poly(2-hydroxyethyl methacrylate), poly(N-(2 hydroxypropyl)methacrylamide), polylactic acid, poly(lactic-co-glycolic acid), poly(ortho esters), poly(2 oxazoline), poly(sebacic acid), poly(terephthalate-co-phosphate), povidone and copolymers.

Preferred hydrophilic polymers include poly(ethylene glycol), hyaluronic acid, dextran, polyethylenimine, poloxamers, povidone, D-alpha-tocopheryl and polyethylene glycol succinate. The molecular weight of the hydrophilic polymer in the polymer-flavonoid conjugate is in general 1K-100K Daltons, preferably 2K-40K, 2K-50K, 2K-80K, 3K-80K, or 5K-40K Daltons.

In one embodiment, the polymer contains an aldehyde group which is conjugated to the 5, 6, 7, or 8 position (preferably 6 or 8 position) of the A ring of the flavonoid compound.

In another embodiment, the polymer contains a thiol group which is conjugated to R₁ or R₂ of the B-ring of a flavonoid (when R₁ or R₂ is —OH).

In one embodiment, the polymer-flavonoid conjugate is PEG-EGCG, which is PEG conjugated to one or two molecules of epigallocatechin gallate (EGCG). PEG-EGCG, for example, can be prepared by conjugating aldehyde-terminated PEG to EGCG by attachment of the PEG via reaction of the free aldehyde group with the 5, 6, 7, or 8 position (preferably 6 or 8 position) of Formula I. See WO2006/124000 and WO2009/054813. PEG-EGCG can also be prepared by conjugating thio-terminated PEG to EGCG by attachment of the PEG via reaction of the free thio group with the R₁ or R₂ of Formula I, wherein, R₁ or R₂ is a phenyl group. See WO2015/171079.

### Flavonoid Oligomers

A flavonoid oligomer is a conjugate of one flavonoid with one or more flavonoids. The flavonoid oligomer can contain the same flavonoid (a homo oligomer) or different flavonoids (a hetero oligomer). Flavonoid oligomers useful for the present invention in general have 2-50 or 2-20, preferably 4-12 flavonoids of one or mixed types.

In some embodiment, a flavonoid oligomer is oligomeric EGC (OEGCG), oligomer EC (OEC), oligomer EGC (OEGC), or oligomer ECG (OECG). OEGCG refers to 3-20 monomers of EGCG that are covalently linked. OEGCG, for example, can be synthesized at 5, 6, 7, or 8 position (preferably 6 or 8 position) of the A ring according to WO2006/124000.

Because A-ring is present in all of the flavonoids according to Formula 1, other oligomeric flavonoids can be made similarly according to WO2006/124000. For example, OEC, OEGC, and OECG can also be made according to WO2006/124000.

### MINC-Agent

MINC (Multi-pathway Immune-modulating Nanocomplex Combination therapy) is a platform technology, utilizing the bioactivity of polymer-flavonoid conjugates or flavonoid oligomers that form micelles in a solution.

MINC platform can encapsulate additional agents to form a nanoparticle composition for a therapy. MINC-agent is a micelle having an outer shell formed by one or more polymer-flavonoid conjugates and optionally an inner shell formed by one or more flavonoid oligomer and a drug encapsulated within the shells. The agent, as used herein, reference to a molecule that have a therapeutic activity (e.g., a drug). For example, the encapsulated agents can be various molecules including small molecules, peptides, proteins, monoclonal antibodies, and vaccine.

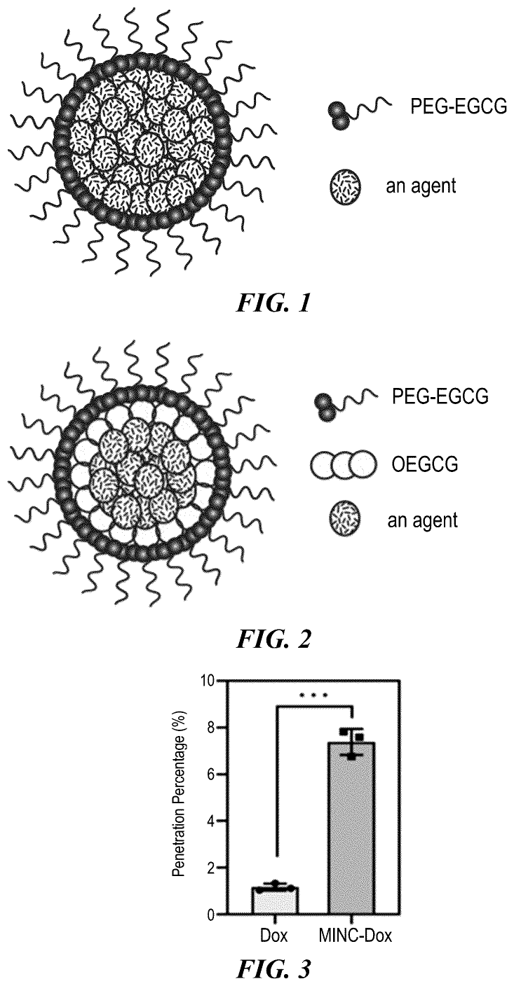

In one embodiment, MINC-agent is a micelle comprises a polymer-flavonoid conjugate, for example, a PEG-EGCG conjugate, in a shell and with an agent encapsulated (see FIG. 1).

In another embodiment, MINC-agent is a micelle comprises a polymer-flavonoid conjugate, for example, a PEG-EGCG conjugate in an outer shell and a flavonoid oligomer, for example, oligomeric EGCG (OEGCG), in an inner shell, with an agent encapsulated (see FIG. 2).

When the agent is a drug, the MINC-Agent composition comprises two or more components that have therapeutic activities, which are complementary in function to form a multiple targeted combination therapy by its backbone components (a flavonoid conjugate or a flavonoid oligomer), and the encapsulated agent.

In one embodiment, the agent in MINC-agent is an antibody including but not limited to anti-HER2, anti-EGFR, anti-PD-L1, anti-PDGFRA, anti-VEGFR2 anti-β amyloid, anti-tau, or anti-α-synuclein.

In one embodiment, the agent in MINC-agent is a cytokine including but not limited to IL-2, IL-4, IL-12, IFN-α, IFN-β, IFN-γ, TNF-α, GM-CSF, GDNF, NRTN, PDGF-BB, CDNF.

In one embodiment, the agent is a small compound including but not restricted to doxorubicin, disulfiram, celecoxib, temsirolimus, everolimus, vorinostat, cabozantinib, marizomib, fimepinostat, acetazolamide, metformin, vinblastine, and cyclophosphamide.

For example, the MINC-agent is anti-HER2 encapsulated in the micelle formed by a polymer-flavonoid conjugate PEG-EGCG and a flavonoid oligomer OEGCG. See WO2009/054813 for the structure and formulation method.

For example, the MINC-agent is doxorubicin encapsulated in the micelle formed by a polymer-flavonoid conjugate PEG-EGCG. See WO2011/112156 for the structure and formulation method.

### Pharmaceutical Compositions

The present invention uses pharmaceutical compositions comprising the polymer-flavonoid conjugate, flavonoid oligomer, or MINC-agent as described in the application, and optionally one or more pharmaceutically acceptable excipients. The nanoparticle component in a pharmaceutical composition in general is about 1-100% or 1-90%, preferably 20-90%, or 30-80% for a tablet, powder, or parenteral formulation. The polymer-flavonoid conjugate, flavonoid oligomer, or MINC-agent composition in a pharmaceutical composition in general is 1-100%, preferably 20-100%, 50-100%, or 70-100% for a capsule formulation. The nanoparticle composition in a pharmaceutical composition in general is 1-50%, 5-50%, or 10-40% for a liquid suspension formulation.

In one embodiment, the pharmaceutical composition can be in a dosage form such as tablets, capsules, granules, fine granules, powders, suspension, solution, patch, parenteral, injectable, or the like. The above pharmaceutical compositions can be prepared by conventional methods.

Pharmaceutically acceptable carriers, which are inactive ingredients, can be selected by those skilled in the art using conventional criteria. The pharmaceutically acceptable carriers may contain ingredients that include, but are not limited to, saline and aqueous electrolyte solutions; ionic and nonionic osmotic agents, such as sodium chloride, potassium chloride, glycerol, and dextrose; pH adjusters and buffers, such as salts of hydroxide, phosphate, citrate, acetate, borate, and trolamine; antioxidants, such as salts, acids, and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants, such as lecithin and phospholipids, including, but not limited to, phosphatidylcholine, phosphatidylethanolamine and phosphatidyl inositol; poloxamers and poloxamines; polysorbates, such as polysorbate 80, polysorbate 60, and polysorbate 20; polyethers, such as polyethylene glycols and polypropylene glycols; polyvinyls, such as polyvinyl alcohol and polyvinylpyrrolidone (PVP, povidone); cellulose derivatives, such as methylcellulose, hydroxypropyl cellulose, hydroxyethyl cellulose, carboxymethyl cellulose, and hydroxypropyl methylcellulose and their salts; petroleum derivatives, such as mineral oil and white petrolatum; fats, such as lanolin, peanut oil, palm oil, and soybean oil; mono-, di-, and triglycerides; polysaccharides, such as dextrans; and glycosaminoglycans, such as sodium hyaluronate. Such pharmaceutically acceptable carriers may be preserved against bacterial contamination using well-known preservatives, which include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet, capsule, or parenteral formulation of the active compound may contain other excipients that have no bioactivity and no reaction with the active compound. Excipients of a tablet or a capsule may include fillers, binders, lubricants and glidants, disintegrators, wetting agents, and release rate modifiers. Examples of excipients of a tablet or a capsule include, but are not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone.

For example, a tablet formulation may contain inactive ingredients, such as colloidal silicon dioxide, crospovidone, hypromellose, magnesium stearate, microcrystalline cellulose, polyethylene glycol, sodium starch glycolate, and titanium dioxide. A capsule formulation may contain inactive ingredients, such as gelatin, magnesium stearate, and titanium dioxide. A powder oral formulation may contain inactive ingredients, such as silica gel, sodium benzoate, sodium citrate, sucrose, and xanthan gum.

The pharmaceutical composition can be applied by local administration and systemic administration. Local administration includes topical administration. Systemic administration includes oral, parenteral (such as intravenous, intramuscular, subcutaneous, or rectal), and other systemic routes of administration. In systemic administration, the active compound first reaches plasma and then distributes into target tissues. Parenteral administration, such as intravenous bolus injection or intravenous infusion, and oral administration are preferred routes of administration.

### Method of Treatment

The present invention is directed to a method of preventing or treating CNS diseases, by administering to a subject in need thereof a polymer-flavonoid conjugate, flavonoid oligomer, or MINC-agent, as described above.

Suitable CNS disease to be treated by the present invention include, but not limited to, neuron degenerative disease, dementia, Alzheimer's disease, Parkinson's disease, Huntington's disease, spongiform encephalopathies, West Nile virus encephalitis, multiple sclerosis, brain injury, spinal cord injury, primary cancer of the brain, and metastatic cancer of the brain, Bell's palsy, headache (largest brain disease), autoimmune disorders, cerebral palsy, motor neuron disease (MND), neurofibromatosis. epilepsy and Seizures, acute spinal cord injury, amyotrophic lateral sclerosis (ALS), ataxia, Bell's Palsy, cerebral aneurysm, obsessive-compulsive disorder (OCD), defects in the cerebral cortex include microgyria, polymicrogyria, bilateral frontoparietal polymicrogyria, and pachygyria.

One important function of polymer-flavonoid conjugate and flavonoid oligomer is to increase drug delivery to the brain (crossing BBB) to enhance therapeutic efficacy. This function is due to the ability of polymer-flavonoid conjugate to penetrate BBB. Drug molecules are encapsulated, and they are not exposed to the BBB and thus they have no influence on entering CNS. This brain delivery applies to all kinds of brain diseases including brain tumors (glioma, choroid plexus tumors, pineal tumors, brain metastatic tumors, meningiomas, pituitary tumors, nerve tumor, central nervous system lymphoma), neuroinflammation diseases (encephalitis, meningitis), neurodegenerative diseases (Alzheimer's disease, Parkinson's disease, Lewy body dementia, Huntington's disease), motor neuron diseases (ataxia, neurofibromatosis, amyotrophic lateral sclerosis (ALS), catalepsy, epilepsy/seizures, locked-in syndrome), CNS diseases with spinal cord injury (acute spinal cord injuries, myelopathy, multiple sclerosis), CNS diseases caused by circulatory system disorder (stroke, cerebral aneurysm), and cerebral cortex disorders (microgyria, polymicrogyria, bilateral frontoparietal polymicrogyria, and pachygyria).

Another function of polymer-flavonoid conjugate and flavonoid oligomer is to reduce neural cell death and enhance cell regeneration to restore cognitive behavior. This function treats neurodegenerative diseases such as Alzheimer's disease, Parkinson's disease, Lewy body dementia, and Huntington's disease. Ex6

Another function of polymer-flavonoid conjugate and flavonoid oligomer is to reduce neural cell oxidative stress and inflammation and to reduce the disease progression and recurrences. Oxidative stress leads to neural toxicity and is involved in the development of neurodegenerative diseases and CNS inflammation diseases including ischemic and hemorrhagic strokes. Reducing neural cell oxidative stress reduces neuronal cell death and treats neuroinflammation diseases (encephalitis, meningitis), motor neuron diseases ALS, locked-in syndrome, catalepsy epilepsy or seizures, CNS diseases with spinal cord injury (acute spinal cord injuries, myelopathy, multiple sclerosis), CNS diseases caused by circulatory system disorder (stroke, cerebral aneurysm), cerebral cortex disorders (microgyria, polymicrogyria, bilateral frontoparietal polymicrogyria, and pachygyria), and neurodegenerative diseases (Alzheimer's disease, Parkinson's disease, Lewy body dementia, Huntington's disease).

Another function of polymer-flavonoid conjugate and flavonoid oligomer is to reduce accumulation of abnormal proteins (e.g., β-amyloid, Tau, and α-synuclein) and to delay and/or reduce the progression and/or recurrence of diseases. These processes treat neurodegenerative diseases (Alzheimer's disease, Parkinson's disease, Lewy body dementia, and Huntington's disease).

Yet another function of polymer-flavonoid conjugate and flavonoid oligomer is to activate patient immune system and reduce the development of brain tumors. These processes are useful to treat brain tumors (glioma, choroid plexus tumors, pineal tumors, brain metastatic tumors, meningiomas, pituitary tumors, nerve tumor, CNS lymphoma).

### Polymer-Flavonoid

In the first aspect of the invention, the method comprises the step of administering an effective amount of a polymer-flavonoid conjugate to a subject in need thereof to treat a CNS disease. The CNS disease is brain tumor, circulatory system disorder, CNS disease with spinal cord injury, neuroinflammation disease, motor neuron disease, or cerebral cortex disorder.

“An effective amount,” as used in this application, is the amount effective to treat a disease by ameliorating the pathological condition or reducing the symptoms of the disease.

The polymer-flavonoid conjugate of the present invention is capable of crossing the Blood-Brain Barrier (BBB) from the circulating blood vessel to the brain. The Polymer-flavonoid conjugate has immune and disease-modulating functions for treating CNS disorders. Additionally, it has neuron cell repair or regeneration activity for treating CNS disorders.

In one embodiment, the flavonoid is EGCG, EC, EGC, or ECG.

In one embodiment, the polymer is a hydrophilic polymer having a molecular weight of 1,000 to 100,000 daltons, and is selected from the group consisting of: PEG, hyaluronic acid, dextran, polyethylenimine, poloxamers, povidone, D-alpha-tocopheryl, and polyethylene glycol succinate.

A preferred polymer-flavonoid conjugate is PEG-EGCG.

In one embodiment, the CNS disease is brain tumor selected from the group consisting of: glioma, brain metastatic tumors, nerve tumors, central nervous system lymphoma, choroid plexus tumors, pineal tumors, meningiomas, and pituitary tumors.

In one embodiment, the CNS disease is a CNS disease caused by circulatory system disorder selected from the group consisting of: stroke and cerebral aneurysm.

In one embodiment, the CNS disease is caused by spinal cord injury selected from the group consisting of: multiple sclerosis, acute spinal cord injuries, and myelopathy,

In one embodiment, the CNS disease is a neuroinflammation disease of encephalitis or meningitis.

In one embodiment, the CNS disease is a motor neuron disease is selected from the group consisting of: amyotrophic lateral sclerosis (ALS), epilepsy, seizures, catalepsy, ataxia, and locked-in syndrome.

In one embodiment, the CNS disease is a cerebral cortex disorder selected from the group consisting of: microgyria, polymicrogyria, bilateral frontoparietal polymicrogyria, and pachygyria.

Dosing for a polymer-flavonoid, e.g., PEG-EGGC, for injection, is in general 0.1-5000 mg/kg (total weight of the polymer-flavonoid/subject body weight), or 1-1000 mg/kg.

### Flavonoid Oligomer

In a second aspect of the invention, the method comprises the step of administering an effective amount of a flavonoid oligomer to a subject in need thereof, to treat a CNS disease. The CNS disease is brain tumor, circulatory system disorder, CNS disease with spinal cord injury, neuroinflammation disease, motor neuron disease, or cerebral cortex disorder.

The flavonoid oligomer of the present invention is capable of crossing the BBB from the circulating blood vessel to the brain. The flavonoid oligomer has immune and disease-modulating functions for treating CNS disorders. Additionally, it has neuron cell repair or regeneration activity for treating CNS disorders.

In one embodiment, the flavonoid oligomer is an oligomer of EGCG, EC, EGC, or ECG.

In one embodiment, the flavonoid oligomer comprises 4-12 flavonoids of EGCG, EC, EGC, or ECG.

In one embodiment, the CNS disease is brain tumor selected from the group consisting of: glioma, brain metastatic tumors, nerve tumors, central nervous system lymphoma, choroid plexus tumors, pineal tumors, meningiomas, and pituitary tumors.

In one embodiment, the CNS disease is a CNS disease caused by circulatory system disorder selected from the group consisting of: stroke and cerebral aneurysm.

In one embodiment, the CNS disease is caused by spinal cord injury selected from the group consisting of: multiple sclerosis, acute spinal cord injuries, and myelopathy,

In one embodiment, the CNS disease is a neuroinflammation disease of encephalitis or meningitis.

In one embodiment, the CNS disease is a motor neuron disease is selected from the group consisting of: amyotrophic lateral sclerosis (ALS), epilepsy, seizures, catalepsy, ataxia, and locked-in syndrome.

In one embodiment, the CNS disease is a cerebral cortex disorder selected from the group consisting of: microgyria, polymicrogyria, bilateral frontoparietal polymicrogyria, and pachygyria.

Dosing for a flavonoid oligomer, e.g., OEGCG, for injection, is in general 0.1-1000 mg/kg (total weight of the flavonoid oligomer/subject body weight), or 1-100 mg/kg.

### MINC-Drug

In a third aspect of the invention, the method comprises the step of administering to a subject in need thereof an effective amount of micelles having an outer shell comprising one or more polymer-flavonoid conjugates and optionally an inner shell comprising one or more flavonoid oligomer and a drug encapsulated within the shells, to treat a CNS disease. In one embodiment, the outer shell is formed by one or more polymer-flavonoid conjugates. In one embodiment, the inner shell is formed by one or more flavonoid oligomer.

The CNS disease is Alzheimer's disease, Parkinson's disease, Lewy body dementia, brain tumors, stroke, Huntington's disease, multiple sclerosis, amyotrophic lateral sclerosis (ALS), or acute spinal cord injury, encephalitis, epilepsy, seizures, meningitis, motor neuron disease (MND), or cerebral aneurysm.

The Polymer-flavonoid conjugate or the flavonoid oligomer is capable of crossing BBB from the circulating blood vessel to the brain as an agent delivery vehicle to deliver said agent for treating or diagnosing CNS disorders.

In one embodiment, the polymer is a hydrophilic polymer having a molecular weight of 1,000 to 100,000 Daltons, and is selected from the group consisting of: poly(ethylene glycol) (PEG), hyaluronic acid, dextran, polyethylenimine, poloxamers, povidone, D-alpha-tocopheryl, and polyethylene glycol succinate;

In one embodiment, the flavonoid oligomer comprises 2-20 flavonoids of EGCG, EC, EGC, or ECG;

In one embodiment, the shell is formed by PEG-EGCG.

In one embodiment, the shell is formed by PEG-EGCG and OEGCG.

In one embodiment, the CNS disease is Alzheimer's disease, and the drug is anti-β amyloid, anti-tau, anti-IL6R, anti-IL-1, anti-CD38, anti-TREM2. or BDNF.

In one embodiment, the CNS disease is Parkinson's disease, and the drug is anti-α-synuclein, anti-IL6R, anti-IL-1, anti-CD38, anti-TREM2, GDNF, NRTN, PDGF-BB, CDNF, or BDNF.

In one embodiment, the CNS disease is Lewy body dementia, and the drug is anti-amyloid or, anti-α-synuclein, anti-IL6R, anti-IL-1β, anti-CD38, anti-TREM2, or BDNF.

In one embodiment, the CNS disease is brain tumor, and the drug is doxorubicin, disulfiram, celecoxib, temsirolimus, everolimus, vorinostat, cabozantinib, marizomib, fimepinostat, acetazolamide, metformin, vinblastine, cyclophosphamide, anti-HER2, anti-EGFR, anti-PD-1, anti-PD-L1, anti-PDGFRA, anti-VEGFR2, IL-2, IL-4, IL-12, IFN-α, IFN-0, IFN-7, or TNF-α.

In one embodiment, the CNS disease is stroke, and the drug is MMP inhibitor, eNOS inhibitor, anti-TLR4, anti-HSP, anti-IL6, anti-IL-12, S1000, Fibronectin, MCP-1, MMP9, UCH-L1, BDNF, GDNF, NRTN, PDGF-BB, or CDNF.

In one embodiment, the CNS disease is Huntington's disease, and the drug is anti-mHtt, anti-α-synuclein, anti-SEMA4D, anti-TNFα, Tetrabenazine, deutetrabenazine, valbenazine, bevantolol, pridopidine, branaplam, nilotinib, mitoconix, or azathioprine

In one embodiment, the CNS disease is multiple sclerosis, and the drug is anti-CD4, anti-IL-17, anti-CD19, anti-CD20, anti-CD25, anti-CD52, anti-RGMA, anti-IL-12, anti-IL-23, anti-α4 integrin, anti-IL-2R, LINGO-1, or anti-NOGO-A.

In one embodiment, the CNS disease is amyotrophic lateral sclerosis (ALS), and the drug is anti-NOGO-A, PKC inhibitor, IGF-1, NOGO-A, GDNF, VEGF, anti-SOD1, SIR, GLT-1, anti-Ataxin2, anti-TDP43, anti-hnRNPs, CK-1 inhibitor, anti-FET or HDAC inhibitor, EPO, or IL-2.

In one embodiment, the CNS disease is acute spinal cord injury, and the drug is Extracellular domain of Nogo receptor, 5-HT1A receptor, FGF, GSK-3b3 inhibitor, anti-IN-1, TNF-α, IL-12, SDF-1α, SOD1, NEC-1, anti-P-selectin, or anti-CD11d.

In one embodiment, the CNS disease is encephalitis, and the drug is anti-FcRn, anti-IL-6, anti-CD20, anti-CD19, anti-CD38, anti-C5, or IL-2.

In one embodiment, the CNS disease is epilepsy or seizures, and the drug is mTOR inhibitor, PI3K inhibitor, GABA inhibitor, anti-Glu3B peptide antibody, anti-NR1 antibody, anti-CASPR2, or anti-LGI-1.

In one embodiment, the CNS disease is meningitis, and the drug is C1 inhibitor, anti-C5, anti-MASP-2, anti-PD-L1, anti-CTLA-4, or anti-PD-1.

In one embodiment, the CNS disease is motor neuron disease (MND), and the drug is Anti-SOD1, anti-TDP-43, anti-C90RF72, anti-Nogo-A, anti-MuSK, anti-IL-6R, anti-NRP-1, anti-Myostatin, anti-CD40L, anti-DR-6, anti-IFN-g, anti-GD1a, anti-CTGF, or anti-HMGB1.

In one embodiment, the CNS disease is cerebral aneurysm, and the drug is TNF-α inhibitor, MMP inhibitor, MCP-1 inhibitor, Phosphodiesterase-4 inhibitor, Mast cell degranulation inhibitor, anti-IL-1beta.

Dosing of the MINC-agent is based on the known dosage of the agents for treating a particular disease and the subject condition. The dosage can be a food drug administration (FDA) approved dosage or a dosage used in clinical trial.

In MINC-agent, in general, the dosage of PEG-EGCG combined with OEGCG is between 10 μg/kg to 100 mg/kg.

The concentration for the encapsulated drug agents can be as low as 10 μg/kg (e. g., for cytokine drugs, rhBDNF) and as high as 100 mg/kg (for antibody drugs, e.g., anti-α synuclein antibody is at this level).

For example, for treating brain metastatic breast cancer in an adult human, anti-HER2 (Trastuzumab) is administered 6-10 mg/kg IV every 3 weeks. The effective dose of MINC-anti-HER2 in the same dose range can be used for treating brain metastatic breast cancer.

For treating glioma, liposome-doxorubicin is given at 40 mg/m² IV every 4 weeks. The effective dose of MINC-doxorubicin in the same dose range can be used for treating glioma.

For treating Alzheimer's disease, anti-β amyloid (Aducanumab) is given at 10 mg/kg IV every four weeks. The effective dose of MINC-anti-β amyloid in the same dose range can be used for treating Alzheimer's disease.

For treating Alzheimer's disease, anti-tau antibody (Semorinemab) is given at 3-30 mg/kg IV every four weeks. The effective dose of MINC-anti-tau in the same dose range can be used for treating Alzheimer's disease.

For treating Parkinson's disease, anti-α synuclein antibody is given between 1500-4500 mg IV every four weeks. The effective dose of MINC-anti-α synuclein in the same dose range can be used for treating Parkinson's disease.

For promoting neuron cell regeneration caused by stroke and neuron degenerative diseases, BDNF (rhBDNF) is given at 25-100 μg/kg intrathecal or IV injection daily. The effective dose of MINC-BDNF in the same dose range can be used for treating stroke and neuron degenerative diseases, which cause neural cell death.

In addition to reducing misfolded protein aggregation in 0-amyloid and tau for Alzheimer's disease, and α-synuclein for Parkinson's disease, EGCG also exerts neuron protective function in these and additional central nerve system diseases. Mechanically, EGCG has the neuron protective functions on neuron cells directly. These include antioxidant activity by working as free radical scavenger and anti-apoptotic activity by reducing expression of proapoptotic genes. The EGCG in the polymer-flavonoid conjugate and MINC-agent provides additional benefits of protecting neural cells from damage by toxins.

The present invention is useful in treating human and non-human animals. For example, the present invention is useful in treating a mammal subject, such as humans, horses, pigs, cats, and dogs.

The present invention provides use of a composition in preventing or treating a CNS disease, or maintaining the health of a CNS subject, wherein the composition comprises (i) a polymer-flavonoid conjugate, (ii) one or more flavonoid oligomers, or (iii) micelles having an outer shell formed by one or more polymer-flavonoid conjugates and optionally an inner shell formed by one or more flavonoid oligomer and a drug encapsulated within the shells, and having an agent encapsulated within the shell, wherein the agent is a drug.

The following examples further illustrate the present invention. These examples are intended merely to be illustrative of the present invention and are not to be construed as being limiting.

### EXAMPLES

### Active Ingredients

### OEGCG:

OEGCG is oligomerized EGCG. OEGCG is prepared according to WO2006/124000.

### PEG-EGCG:

PEG-EGCG is PEG conjugated with one or two EGCG. PEG-EGCG is prepared according to WO2006/124000, WO2009/054813, or WO2015/171079.

### MINC-Doxorubicin:

MINC-doxorubicin is doxorubin encapsulated within PEG-EGCG and it is prepared according to WO2011/112156.

### Other MINC-Agents:

MINC-agents are made according to WO2011/112156 or WO2015/171079. Alternatively, MINC-agents can be prepared by encapsulated an agent within the micelle formed by PEG-EGCG and OEGCG, according to the method in WO2006/124000 or WO2009/054813.

### MINC-Drug Brings Drugs Through BBB and to Treat CNS Diseases (Examples 1-3)

### Example 1: MINC-Doxrubicin Delivers Doxorubicin to the Brain in Zebrafish

### Materials

OEGCG is oligomerized EGCG. OEGCG is prepared according to WO2006/124000.

PEG-EGCG is PEG conjugated with one or two EGCG. PEG-EGCG is prepared according to WO2006/124000, WO2009/054813, or WO2015/171079.

MINC-doxorubicin is doxorubicin encapsulated within PEG-EGCG and it was prepared according to WO2011/112156.

Zebrafish (Zebrafish International Resource Center, University of Oregon, USA).

### Method

Doxorubicin does not efficiently penetrate BBB into brain parenchyma. We formulated MINC-doxorubicin and a zebrafish model was used to test whether MINC formulation can bring the encapsulated doxorubicin through BBB to the brain.

Briefly, Tg(fli1a:EGFP) transgenic zebrafish obtained from the Zebrafish International Resource Center were kept under a 14-hour light and 10-hour dark photoperiod at 28.5° C. Following fertilization, the eggs were collected and cultured in an aquarium. Embryos developed to the 48 h post-fertilization (hpf) stage were used for cardiac venous sinus microinjection. For microinjection, MINC-doxorubicin was diluted to 386.37 μM in ddH2O containing phenol red (1%) as an injection tracer. Same amount of doxorubicin was used as control. After injecting MINC-doxorubicin (doxorubicin is a red fluorescent compound) using NANOLITER 2000 microinjector with Micromanipulators 3301R manipulator, the distribution of doxorubicin (red fluorescence) was observed under Leica DM 2500 fluorescence microscope using filter CHROMA 41004 (mcherry).

### Results

Under the fluorescent microscope, it was shown that MINC platform delivers more doxorubicin to the brain than the control doxorubicin (without MINC).

This experiment used a transparent zebrafish model in order to visualize a red colored drug doxorubicin. which is not able to enter the brain. Doxorubicin was able to enter the brain in large quantity (bright red) after MINC formulation.

### Example 2: MINC Platform Delivers Drug to Penetrate BBB in Surrogate Cell Model

### Materials

OEGCG, PEG-EGCG, and MINC-Doxorubin are the same as described in Example 1.

### Method

To confirm the efficacy of MINC platform in bringing drugs across blood brain barrier, we select doxorubicin for in vitro BBB transwell study.

In brief, 3×10⁴ Caco-2 cells were seeded into the luminal side of the insert of 24 well transwell plate (Falcon). Medium was changed every 3 days. Transepithelial electrical resistance (TEER) value which indicated the BBB barrier integrity of each well is measured by a Millicell ERS Voltohmmeter (Millipore, MA, USA). When the TEER value of each insert reached 250 Ω*cm², either (1) 5 μg/mL unencapsulated free doxorubicin or (2) MINC-doxorubicin with fluorescent intensity equivalent to 5 μg/mL doxorubicin were added to the top of inserts. After 8 hours of incubation, medium in the upper insert and the lower culture well was collected and fluorescence signal (relative fluorescence unit, RFU) at Ex/Em=470/595 nm was detected by Spectramax i3x. Drug penetration percentage was calculated following the formula:

$$penetration ( \% ) = ( R F {U}_{L o w e r} \times 7 ) \div ( {RFU}_{Upper} \times 7 + R F {U}_{L o w e r} \times 7 )$$

### Results

The results are shown in FIG. 3: more fluorescence signals were shown in MINC-doxorubicin treatment group compared with doxorubicin group. The results demonstrate that more MINC-doxorubicin penetrated the surrogate BBB transwell model than doxorubicin alone.

### Example 3: MINC Platform Delivers Anti-HER2 to the Brain in Mouse

### Materials

OEGCG and PEG-EGCG are described as in Example 1.

Cyanine5.5 NHS ester (Cy 5.5) (Aladdin)

Anti-HER2-Cy5.5 conjugate is prepared by reacting the anti-HER2 antibody with Cy5.5-NHS ester according to manufacturer's instructions (Aladdin).

MINC-anti-HER2-Cy5.5 is anti-HER2-Cy5.5 encapsulated within PEG-EGCG and OEGCG, and it is prepared according to WO2009/054813.

### Method

Antibody drugs cannot penetrate BBB into brain parenchyma efficiently. We used fluorophore-labeled anti-HER2 (trastuzumab) as an example to demonstrate that MINC formulation can bring antibody drugs to the brain in mouse model.

Athymic Nude-Foxn1^{nu} female mice at the age of 6 weeks were used, the mice were divided into two groups. One group (n=3) received 10 mg/kg anti-HER2-Cy5.5 through tail vein i.v. bolus as control. The other group (n=3) received equivalent anti-HER2-Cy5.5 dose in MINC-anti-HER2-Cy5.5 via tail vein i.v. bolus. After drug administration, the live images were observed at 8 hour, with ex/em of 674/692 nm, using an IVIS (in vivo imaging system) Lumina III XRMS.

### Results

The results showed that observed that fluorescence signal is present in the brain region of mice receiving MINC-anti-HER2-Cy5.5 (STM-001) but not in the mice treated with anti-HER2-Cy5.5 (trastuzumab).

Trastuzumab is able to treat HER2+ breast cancer, but it is not approved for glioma because poor delivery into the brain. This example shows MINC-trastuzumab can deliver trastuzumab into the brain in mouse.

Flavonoid-Oligomer (Example 4) and MINC-Agent (Examples 5-6) have Efficacies in Treating Hard to Treat CNS Diseases

### Example 4: OEGCG Suppresses Glioma Cell Growth

### Materials

OEGCG is prepared according to Example 1.

CCK-8 kit (Targetmol, Shanghai, China)

A172 cell line (HER2+ glioma cell line) (CRL-1620, ATCC)

U87 cell line (HER2− glioma cell line) (HTB-14, ATCC)

### Method

We used in vitro tumor suppression assay to study the effect of OEGCG in glioma growth. Both HER2 positive and negative human glioma cell lines A172 (HER2+) and U87 (HER2−) obtained from ATCC were used to test the anti-tumor ability of OEGCG on glioma. Temozolomide-resistant cells derived from A172 and U87 were used to investigate potential of OEGCG to overcome temozolomide resistance. In brief, cells were seeded in 96-well plates at a density of 5000 cells/well and were allowed to adhere to the surface for 24 hours. Cells were then treated with OEGCG at 0, 30, 60, 120 μM for 72 h. Following treatment, the treated cells were incubated with CCK-8 reagent for 1 h at 37° C., and the absorption values were detected at 450 nm using a SpectraMax® iD3 reader. The results were reported as the mean±standard deviation of at least two replicates.

### Results

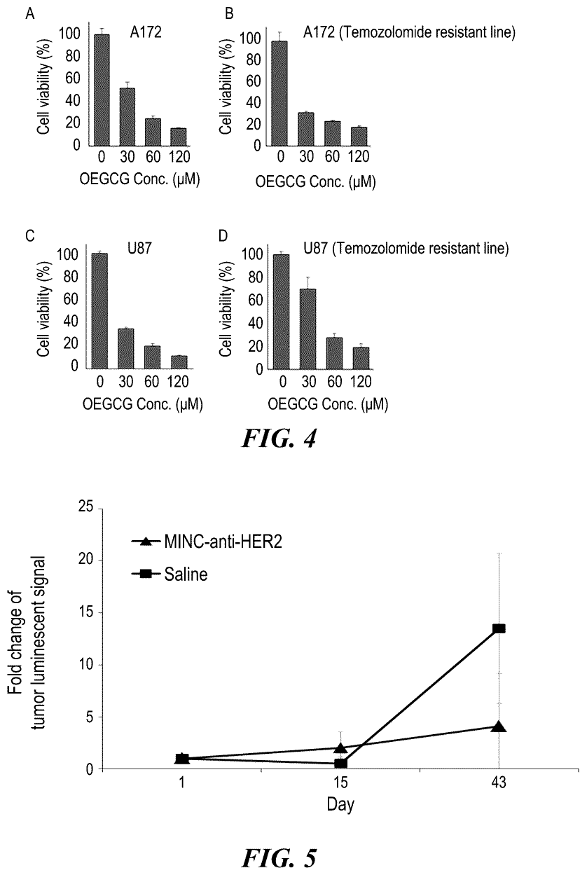

FIG. 4 shows the tumor suppression efficacy of OEGCG in two glioma cell lines, A172 (HER2+) and U87 (HER2−). The results demonstrate that OEGCG has broad efficacy for treating glioma cells regardless of their HER2 expression level (FIGS. 4A and 4C). In addition, OEGCG is also effective for treating temozolomide resistant glioma cells (FIGS. 4B and 4D). Temozolomide is first line treatment for high grade glioma, the results support that OEGCG can overcome temozolomide resistance and be used as a combination therapy with additional drugs encapsulated in MINC.

### Example 5: MINC-Anti-HER2 Efficacy Study in Glioma Mouse Model

### Materials

OEGCG and PEG-EGCG are prepared according to Example 1.

MINC-anti-HER2-Cy5.5 is anti-HER2 encapsulated within PEG-EGCG and OEGCG, and it is prepared according to WO2009/054813.

A172 glioma cell line (CRL1620, ATCC); D-Luciferin (Sigma-Aldrich)

### Method

We used an orthotopic glioma mouse model to confirm the efficacy of MINC-anti-HER2 in treating glioma. For imaging tumor size, A172 cell line was engineered with luciferase genes (A172-Luc). For the generation of the mouse model, a skull burr hole was created in the right frontal brain area. An ultrafine needle was then inserted to a depth of 3 mm using a stereotactic guiding device, and then, 1×10⁶ A172-Luc cells (suspended in 3 μl of DMEM) were injected slowly to the mouse brain. The mice were divided into two groups, vehicle group (saline as no treatment control) and MINC-anti-HER2 group.

Two weeks after tumor implantation, saline or MINC-anti-HER2 was tail vein i.v. injected at 10 mg/kg twice per week for 43 days. Tumor size was examined using an in vivo imaging system (IVIS) biweekly. After the mice were anesthetized, mice were injected with luciferin solution intraperitoneally and then transferred to the IVIS chamber for image acquisition.

### Results

FIG. 5 shows that comparing to the saline treatment control group, mice treated with MINC-anti-HER2 have reduced luciferase signal in A172 glioma. The data demonstrate that MINC-anti-HER2 effectively suppressed glioma growth.

### Example 6: OEGCG and MINC-BSA Suppress Triple Negative Cancer Cell Growth

### Materials

OEGCG is prepared according to Example 1.

MINC-BSA is BSA encapsulated within PEG-EGCG and OEGCG, and it is prepared according to WO2009/054813.

Acid Phosphatase (ACP) Assay kit (LSBio).

MDA-MB-231 cell line (CRM-HTB-26D, ATCC).

MDA-MB-468 cell line (HTB-132, ATCC).

BT-20 cell line (HTB-19, ATCC).

### Method

We used in vitro tumor suppression assay to study the effect of OEGCG and MINC-BSA in triple negative breast cancer. Human MDA-MB-231, MDA-MB-468 and BT-20 triple negative breast cell lines obtained from ATCC were used. In brief, cells were seeded in 96-well plates at a density of 3,000 to 10,000 cells/well and were allowed to adhere to the surface for 24 hours. Cells were then treated with OEGCG at 0, 256, 320, 400 and 500 μM for 72 h or MINC-BSA at 0, 4.2, 8.3 and 16.7 μM for 72 h. Following treatment, the treated cells were incubated with pNPP reagent for 30 mins at 37° C., and the absorption values were detected at 410 nm using a SpectraMax® iD3 reader. The results were reported as the mean±standard deviation of at least three replicates. Acid Phosphatase (ACP) is an enzyme which catalyzes the cleavage of phosphate groups from other molecules during digestion.

The enzyme levels can be used as a biomarker for cancer. This non-radioactive, colorimetric ACP assay is based on the cleavage of p-Nitrophenol from the synthetic substrate. The increase in absorbance at 405 nm after addition of the stop reagent is directly proportional to the enzyme activity, reflecting the viability of cancer cells.

### Results

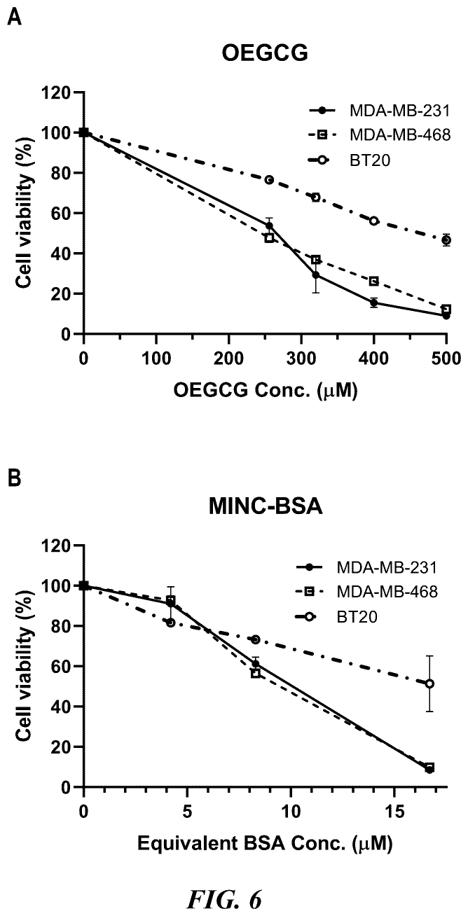

FIG. 6 shows the tumor suppression efficacy of OEGCG and MINC-BSA in three triple negative breast cancer cell lines. The results demonstrate that OEGCG (FIG. 6A) and MINC-BSA (FIG. 6B) have significant tumor suppression effect in triple negative breast cancer cell lines. Triple negative breast cancer are non-responder to anti-HER2 therapy. These results support that OEGCG and MINC nanoparticle platform (MINC-BSA, BSA has no anticancer effect by itself) can overcome anti-HER2 therapy resistance, and MINC can be used with additional drugs encapsulated. Considering that 25-46% of triple negative breast cancer patients have brain metastasis issue, the results also indicate MINC platform can be used for the brain metastatic cancer patients.

### Alzheimer's Disease (Examples 7-8)

### Example 7. OEGCG Suppresses Aβ Induced Cell Death

### Materials

OEGCG is prepared according to Example 1.

Recombinant Aβ (1-42) peptide, MTT (3-(4,5-Dimethylthiazol-2-yl)-2,5-Diphenyltetrazolium Bromide) are purchased from Genscript, Thermo Fisher, or any publically available suppliers. HT-22 cell was obtained from Millipore (Bedford, MA, USA)

### Method

To confirm the efficacy of OEGCG in protecting Aβ-induced neural cell death, an in vitro MTT assay was conducted. In brief, HT-22 cells were seed 2*10⁵ per well in 24-well dish and maintained in a logarithmic growth phase for 3 days. The cell then treated with prepared 2.5 M oligo-Aβ, Aβ+OEGCG for 24 hours. After incubation, cells were washed once with warmed PBS to remove test materials and add tetrazolium salt for 30 mins at room temperature. Then the formazan product was measured spectrophotometrically at 550 nm. Viability was calculated as percent of control cells treated with vehicle alone (mock).

### Result

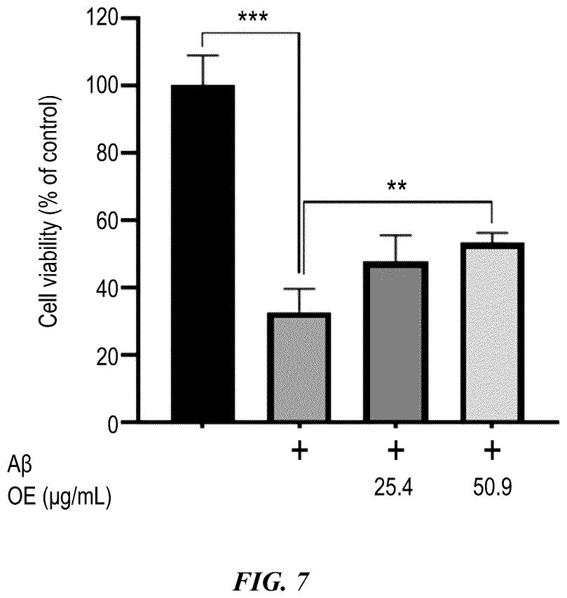

FIG. 7 shows the ability of OEGCG in protecting Aβ-induced cell death. Pathogenic Aβ accumulation can induce neural cell death and lead to neurodegeneration. Reduction of Aβ accumulation have therapeutic potential to treat Alzheimer's disease. The result demonstrates OEGCG reduces Aβ-induced cell death, which is the cause and primary target to treat Alzheimer's disease.

### Example 8. OEGCG and PEG-EGCG Suppress Aβ-Induced Oxidative Stress

### Materials

OEGCG and PEG-EGCG are prepared according to Example 1.

Recombinant Aβ (1-42) peptide, DCFH-DA are purchased from Genscript, Thermo Fisher, or any publically available suppliers.

HT-22 cell was obtained from Millipore (Bedford, MA, USA)

### Method

To confirm the efficacy of OEGCG and PEG-EGCG in reducing Aβ-induced oxidative stress. In vitro cell reactive oxygen species (ROS) staining test was conducted. HT-22 cells were seeded 2×105 cells per well in 24-well dish and maintained for 3 days. Then cells were treated with Aβ with or without OEGCG (OE) and PEG-EGCG (PE). Oligo-Aβ was prepared according to previous experience. In brief, Aβ peptide was dissolved in 1 mM in 100% 1,1,1,3,3,3-hexafluoro-2-propanol and was dried using a vacuum desiccator. Next, Aβ was resuspended to a concentration of 5 mM in dimethylsulfoxide (DMSO) and stored at −20° C. To obtain oligomers, Aβ peptide was diluted to a final concentration of 100 M by using Dulbecco's modified Eagle's medium (DMEM; Gibco), incubated at 4° C. after 24 h of gentle shaking, and immediately added to cell cultures at a final concentration of 2.5 M. After cells were treated with Aβ for 1 hour, 50.9, 25.4 and 12.7 μg/mL of OEGCG and 105.7, 52.4 and 26.4 μg/mL of PEG-EGCG were added to cell and incubated for 6 hours. Then cells were treated with 20 M of DCFH-DA for 0.5 h at 37° C. under 5% C02. After DCFH-DA staining, cells were washed twice with DMEM and once with phosphate-buffered saline to remove background signals. The fluorescent images were also collected by fluorescence microscopy (DP72/CKX41, Olympus), and all images were used the same fluorescent conditions and exposure time.

### Result

Aβ induces oxidative stress in neural cells, therefore more reactive oxygen species (ROS) signal can be observed by DCFH-DA staining (green). The results of the fluorescent images show that both OEGCG and PEG-EGCG significantly reduced ROS production. ROS production is the risk factor to induce brain inflammation and cell damage. The results showed that OEGCG and PEG-EGCG have therapeutic potential to protect neuronal cell from Aβ-induced oxidative stress, the cause of neuron death during Alzheimer's disease progression.

### Evidence of Different Flavonoid Oligomers and Polymer-Flavonoid Conjugates in Forming MINC-Drugs (Examples 9-12)

### Example 9: Method for Preparing MINC-Anti-HER2 Using Different Flavonoids in Flavonoid Oligomers and Polymer-Flavonoid Conjugates

### Materials

OEGCG is oligomerized EGCG. OECG is oligomerized ECG. These flavonoid oligomers were prepared according to WO2006/124000.

PEG-EGCG is PEG conjugated with one or two EGCG. PEG-EC is PEG conjugated with one or two EC. PEG-ECG is PEG conjugated with one or two ECG. These polymer-flavonoids were prepared according to WO2006/124000, WO2009/054813, or WO2015/171079.

Anti-HER2 is trastuzumab obtained from Eirgenix.

### Method

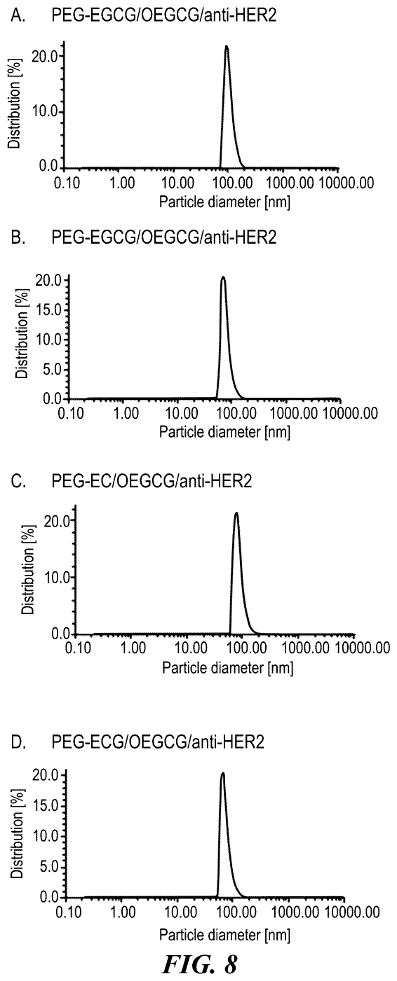

MINC anti-HER2 nanoparticles were prepared according to WO2009/054813. In brief, anti-HER2 was incubated in PBS. Subsequently, different flavonoid oligomer including OEGCG or OECG was added to anti-HER2, followed by adding different polymer-flavonoid including PEG-EGCG, PEG-ECG or PEG-EC. After incubating the mixture at room temperature, 10K MWCO centrifugal filter was used to remove the unreacted oligomer flavonoid and polymer-flavonoid. DLS (Anton Paar Litesizer 500) was used to measure the nanoparticle size and the results are shown in FIG. 8.

### Results

FIG. 8 shows that different polymer-flavonoid conjugate and different flavonoid oligomers all successfully generated MINC-anti-HER2 micelles with one single peak of similar particle size around 100 nm. The results demonstrate that homogenous nanoparticles (micelles) were successfully formed with an expected size, and there was no small peak (about 5-10 nm) of unencapsulated anti-HER2 observed.

In this example, different flavonoid oligomers used include OEGCG (FIGS. 8A, 8C and 8D) and OECG (FIG. 8B); and different polymer-flavonoids used include PEG-EGCG (FIGS. 8A and 8B), PEG-EC (FIG. 8C) and PEG-ECG (FIG. 8D).

These data support that MINC nanoparticle can be formed by different flavonoid oligomers and different polymer-flavonoid conjugates.

### Example 10: Method for Preparing MINC-BSA Using Different Polymers in Polymer-Flavonoid Conjugates

### Materials

OEGCG is oligomerized EGCG, which is prepared according to WO2006/124000.

PEG-EGCG is PEG conjugated with one or two EGCG. HA-EGCG is HA conjugated with one or two EGCG. Dextran-EGCG is Dextran conjugated with one or two EGCG. These different polymer-flavonoids are prepared according to WO2006/124000, WO2009/054813, or WO2015/171079.

BSA is purchased from Sigma-Aldrich.

### Method

MINC (Multi-target Immune Nanocarrier Combination)-BSA nanoparticles were prepared according to WO2009/054813. In brief, BSA was incubated in PBS. Subsequently, OEGCG or OEGCG was added to BSA, followed by adding different polymer-flavonoid including PEG-EGCG, HA-EGCG and Dextran-EGCG. After incubating the mixture at room temperature, 10K MWCO centrifugal filter was used to remove the unreacted OEGCG and polymer-flavonoid. DLS (Anton Paar Litesizer 500) was used to measure the nanoparticle size.

### Results

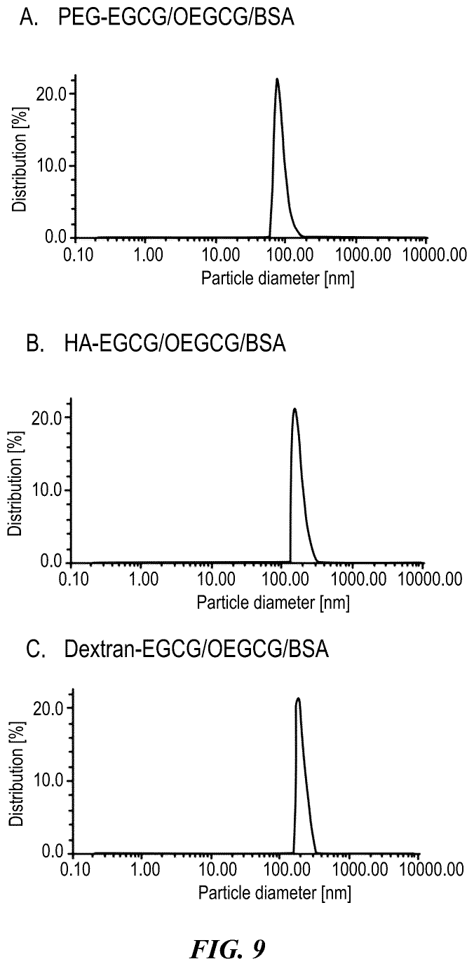

FIG. 9 demonstrates that different polymers in polymer-flavonoid conjugates can be used to successfully generate MINC-BSA. The results demonstrate that homogenous nanoparticles (micelles) were successfully formed, and there was no small peak (about 5-10 nm) of unencapsulated BSA observed. These different polymers are PEG (FIG. 9A), HA (FIG. 9B) and Dextran (FIG. 9C). Altogether, these data support that MINC nanoparticle can be formed by different polymer-flavonoids conjugates.

### Example 11: Method for Preparing MINC-Anti-Aβ

### Materials

OEGCG is oligomerized EGCG, which is prepared according to WO2006/124000.

PEG-EGCG is PEG conjugated with one or two EGCG, which is prepared according to WO2006/124000, WO2009/054813, or WO2015/171079.

Anti-Aβ is purchased from Biolegend.

### Method

MINC-anti-Aβ nanoparticles (PEG-EGCG, OEGCG and anti-Aβ) were prepared according to Example 9. DLS (Anton Paar Litesizer 500) was used to measure the size of MINC-anti-Aβ nanoparticles

### Results

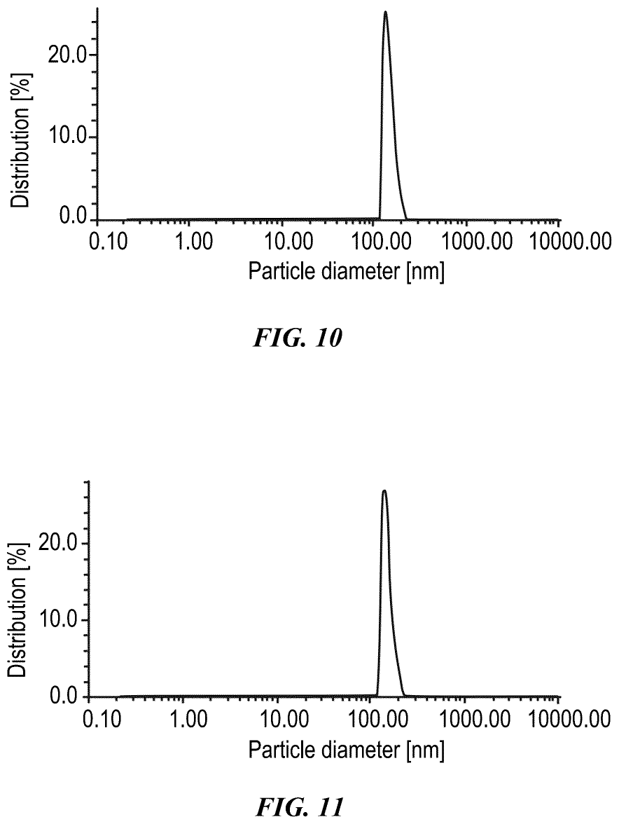

FIG. 10 shows a successful formulation of MINC-anti-Aβ.

### Example 12: Method for Preparing MINC-Anti-α-Syn

### Materials

OEGCG is oligomerized EGCG, which is prepared according to WO2006/124000.

PEG-EGCG is PEG conjugated with one or two EGCG, which is prepared according to WO2006/124000, WO2009/054813, or WO2015/171079.

Anti-α-syn is purchased from Biolegend.

### Method

MINC-anti-α-syn nanoparticles (PEG-EGCG, OEGCG and anti-α-syn) were prepared according to Example 8. DLS (Anton Paar Litesizer 500) was used to measure the size of MINC-anti-α-syn nanoparticles

### Results

FIG. 11 shows a successful formulation of MINC-anti-α-syn.

### LIST OF ABBREVIATIONS

ALS Amyotrophic Lateral Sclerosis
Aβ β-amyloid
α-syn α-synuclein
BBB blood brain barrier
BDNF Brain-Derived Neurotrophic Factor
C1 Complement Component 1
C5 Complement Component 5
CASPR2 Contactin-Associated Protein-Like 2
CDNF Cerebral Dopamine Neurotrophic Factor
CNS Central Nervous System
CTGF Connective Tissue Growth Factor
DR-6 Death Receptor 6
EGFR Epidermal Growth Factor Receptor
eNOS Endothelial Nitric Oxide Synthase
EPO Erythropoietin
FcRn Neonatal Fe Receptor
FET Field-Effect Transistor Antibody
FGF Fibroblast Growth Factor
GABA Gamma-Aminobutyric Acid
GDNF Glial Cell Line-Derived Neurotrophic Factor
GLT1 Glutamate Transporter-1
Glu3B Glutamate [NMDA]Receptor Subunit 3B
GSK-3p inhibitor Glycogen Synthase Kinase-3 Beta Inhibitor
HA Hyaluronic acid
HDAC inhibitor Histone Deacetylases Inhibitor
HER2 Human Epidermal Growth Factor Receptor 2
HMGB1 High Mobility Group Box 1
hnRNPs Heterogeneous Nuclear Ribonucleoproteins
HSP Heat Shock Protein Antibody
HT1A receptor Hydroxytryptamine Receptor 1a
IFN-α Interferon-Alpha
IL-10 Interleukin-1B
IL6R Interleukin-6 Receptor
LGI-1 Leucine-Rich, Glioma Inactivated 1
LINGO-1 Leucine Rich Repeat And Ig Domain Containing 1
MASP-2 Mannan-Binding Lectin Serine Protease 2
MCP-1 Monocyte Chemoattractant Protein-1
MINC Multi-pathway Immune-modulating Nanocomplex Combination therapy
mHtt Mutant Huntingtin Protein
MMP Matrix Metalloproteinase
MMP9 Matrix Metalloproteinase 9
MND Motor Neuron Disease
mTOR Mammalian Target Of Rapamycin
NOGO-A Neurite Outgrowth Inhibitor A
NR1 N-Methyl-D-Aspartate Receptor Subunit 1
NRTN Neurturin
PD-1 Programmed Death-1
PDGF-BB Platelet-Derived Growth Factor-Bb
PDGFRA Platelet-Derived Growth Factor Receptor A
PD-L1 Programmed Death-Ligand 1
PEG Polyethylene glycol
PI3K Phosphoinositide 3-Kinases
RGMA Repulsive Guidance Molecule Bmp Co-Receptor A
SiR Sigma-1 Receptor
SEMA4D Semaphorin 4D
SOD1 Superoxide Dismutase 1
TLR4 Toll-Like Receptor 4 Antibody
TNFα Tumor Necrosis Factor Alpha
TREM2 Triggering Receptor Expressed On Myeloid Cells 2
UCH-L1 Ubiquitin C-Terminal Hydrolase L1
VEGFR2 Vascular Endothelial Growth Factor Receptor 2

The invention, and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as the invention, the following claims conclude this specification.

What is claimed is:

1. A method of treating a central nervous system (CNS) disease, comprising the step of administering to a subject in need thereof an effective amount of micelles having an outer shell comprising one or more polymer-flavonoid conjugates, optionally an inner shell comprising one or more flavonoid oligomer, and a drug encapsulated within the shells;
wherein the polymer is a hydrophilic polymer having a molecular weight of 1,000 to 100,000 daltons, and is selected from the group consisting of: poly(ethylene glycol) (PEG), hyaluronic acid, dextran, polyethylenimine, poloxamers, povidone, D-alpha-tocopheryl, and polyethylene glycol succinate;
the flavonoid is EGCG, EC, EGC, or ECG, as shown in the structures below: 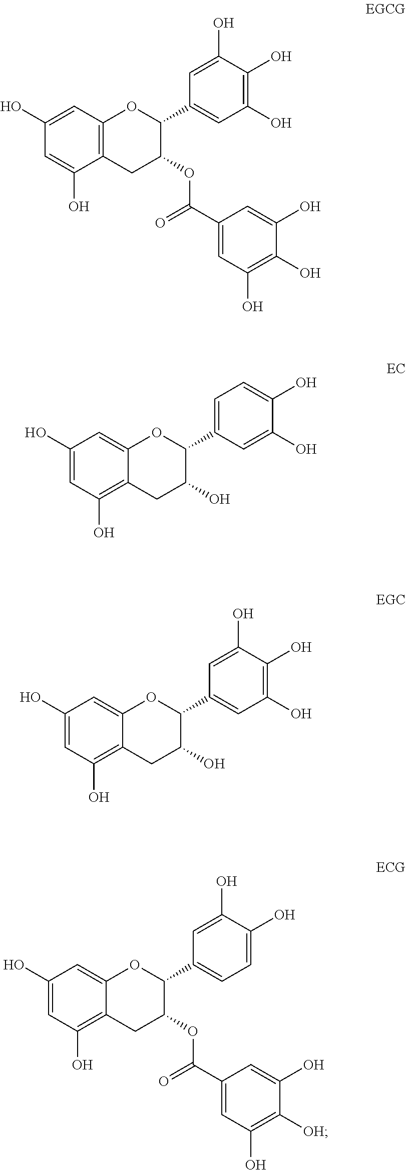
the flavonoid oligomer comprises 2-20 flavonoids of EGCG, EC, EGC, or ECG;
the CNS disease is selected from the group consisting of: Alzheimer's disease, Parkinson's disease, Lewy body dementia, brain tumors, stroke, Huntington's disease, multiple sclerosis, amyotrophic lateral sclerosis (ALS), acute spinal cord injury, encephalitis, epilepsy, seizures, meningitis, motor neuron disease (MND), and cerebral aneurysm.

2. The method according to claim 1, wherein the micelles having an outer shell comprising PEG-EGCG and an inner shell comprising EGCG oligomers.

3. The method according to claim 1, wherein the CNS disease is Alzheimer's disease, and the drug is anti-β amyloid, anti-tau, anti-IL6R, anti-IL-1β, anti-CD38, anti-TREM2, or BDNF.

4. The method according to claim 1, wherein the CNS disease is Parkinson's disease, and the drug is anti-β-synuclein, anti-IL6R, anti-IL-1β, anti-CD38, anti-TREM2, GDNF, NRTN, PDGF-BB, CDNF, or BDNF.

5. The method according to claim 1, wherein the CNS disease is Lewy body dementia, and the drug is anti-β amyloid or, anti-α-synuclein, anti-IL6R, anti-IL-1β, anti-CD38, anti-TREM2, or BDNF.

6. The method according to claim 1, wherein the CNS disease is brain tumor, and the drug is doxorubicin, disulfiram, celecoxib, temsirolimus, everolimus, vorinostat, cabozantinib, marizomib, fimepinostat, acetazolamide, metformin, vinblastine, cyclophosphamide, anti-HER2, anti-EGFR, anti-PD-1, anti-PD-L1, anti-PDGFRA, anti-VEGFR2, IL-2, IL-4, IL-12, IFN-α, IFN-β, IFN-γ, or TNF-α.

7. The method according to claim 1, wherein the CNS disease is stroke, and the drug is MMP inhibitor, eNOS inhibitor, anti-TLR4, anti-HSP, anti-IL6, anti-IL-12, S1000, Fibronectin, MCP-1, MMP9, UCH-L1, BDNF, GDNF, NRTN, PDGF-BB, or CDNF.

8. The method according to claim 1, wherein the CNS disease is Huntington's disease, and the drug is anti-mHtt, anti-α-synuclein, anti-SEMA4D, anti-TNFα, Tetrabenazine, deutetrabenazine, valbenazine, bevantolol, pridopidine, branaplam, nilotinib, mitoconix, or azathioprine.

9. The method according to claim 1, wherein the CNS disease is multiple sclerosis, and the drug is anti-CD4, anti-IL-17, anti-CD19, anti-CD20, anti-CD25, anti-CD52, anti-RGMA, anti-IL-12, anti-IL-23, anti-α4 integrin, anti-IL-2R, LINGO-1, or anti-NOGO-A.

10. The method according to claim 1, wherein the CNS disease is amyotrophic lateral sclerosis (ALS), and the drug is anti-NOGO-A, PKC inhibitor, IGF-1, NOGO-A, GDNF, VEGF, anti-SOD1, SiR, GLT-1, anti-Ataxin2, anti-TDP43, anti-hnRNPs, CK-1 inhibitor, anti-FET or HDAC inhibitor, EPO, or IL-2.

11. The method according to claim 1, wherein the CNS disease is acute spinal cord injury, and the drug is Extracellular domain of Nogo receptor, 5-HT1A receptor, FGF, GSK-3b3 inhibitor, anti-IN-1, TNF-α, IL-12, SDF-1α, SOD1, NEC-1, anti-P-selectin, or anti-CD11d.

12. The method according to claim 1, wherein the CNS disease is encephalitis, and the drug is anti-FcRn, anti-IL-6, anti-CD20, anti-CD19, anti-CD38, anti-C5, or IL-2.

13. The method according to claim 1, wherein the CNS disease is epilepsy or seizures, and the drug is mTOR inhibitor, PI3K inhibitor, GABA inhibitor, anti-Glu3B peptide antibody, anti-NR1 antibody, anti-CASPR2, or anti-LGI-1.

14. The method according to claim 1, wherein the CNS disease is meningitis, and the drug is C1 inhibitor, anti-C5, anti-MASP-2, anti-PD-L1, anti-CTLA-4, or anti-PD-1.

15. The method according to claim 1, wherein the CNS disease is motor neuron disease (MND), and the drug is Anti-SOD1, anti-TDP-43, anti-C90RF72, anti-Nogo-A, anti-MuSK, anti-IL-6R, anti-NRP-1, anti-Myostatin, anti-CD40L, anti-DR-6, anti-IFN-g, anti-GD1a, anti-CTGF, or anti-HMGB1.

16. The method according to claim 1, wherein the CNS disease is cerebral aneurysm, and the drug is TNF-α inhibitor, MMP inhibitor, MCP-1 inhibitor, Phosphodiesterase-4 inhibitor, Mast cell degranulation inhibitor, anti-IL-1beta.

17. A method of treating a CNS disease, comprising the step of administering to a subject in need thereof an effective amount of a polymer-flavonoid conjugate,
wherein the flavonoid is EGCG, EC, EGC, or ECG, as shown in the structures below: 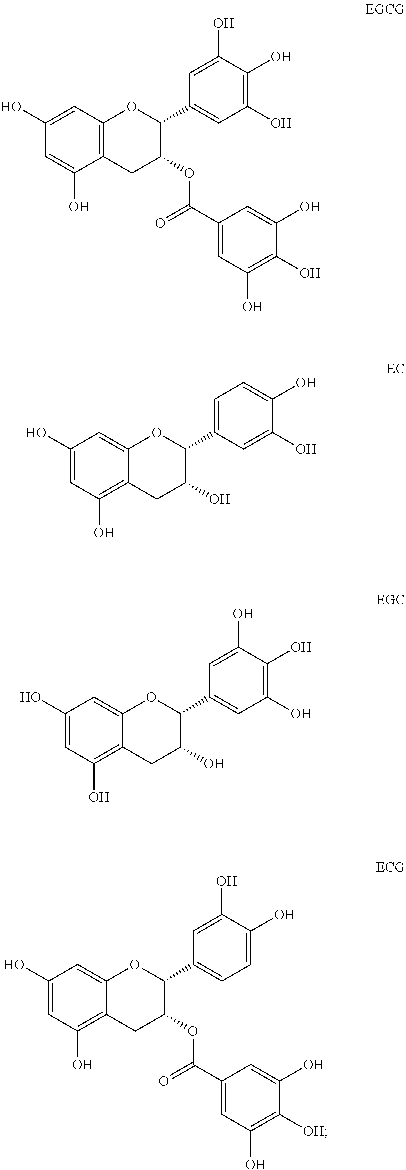
the polymer is a hydrophilic polymer having a molecular weight of 1,000 to 100,000 daltons, and is selected from the group consisting of: PEG, hyaluronic acid, dextran, polyethylenimine, poloxamers, povidone, D-alpha-tocopheryl, and polyethylene glycol succinate; and
the CNS disease is selected from the group consisting of: brain tumor, circulatory system disorder, CNS disease with spinal cord injury, neuroinflammation disease, motor neuron disease, and cerebral cortex disorder.

18. The method according to claim 17, wherein the polymer-flavonoid conjugate is PEG-EGCG.

19. A method of treating a CNS disease, comprising the step of administering to a subject in need thereof an effective amount of a flavonoid oligomer,
wherein the flavonoid is EGCG, EC, EGC, or ECG, as shown in the structures below: 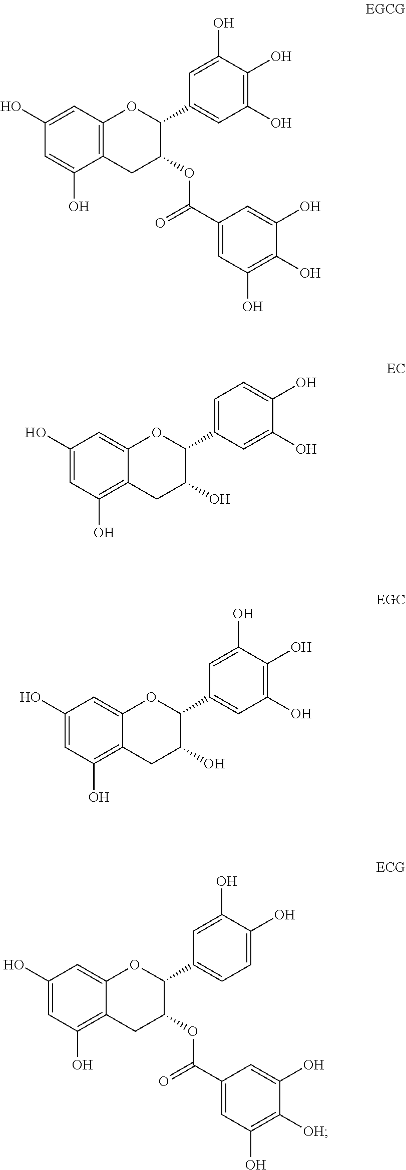
the flavonoid oligomer comprises 4-12 flavonoids of EGCG, EC, EGC, or ECG; and
the CNS disease is selected from the group consisting of: brain tumor, circulatory system disorder, CNS disease with spinal cord injury, neuroinflammation disease, motor neuron disease, and cerebral cortex disorder.

20. The method of claim 19, wherein the flavonoid oligomer is oligomer of EGCG.
